(19) United States
(12) Patent Application Publication
(10) Pub. No.: US 2022/0054539 A1
(43) Pub. Date: Feb. 24, 2022

(54) DISINFECTION AND DEODORIZING OF PAP EQUIPMENT USING LOW CONCENTRATION HYPOCHLOROUS ACID SOLUTIONS

(71) Applicant: Wonder Spray, LLC, San Diego, CA (US)

(72) Inventors: John Burd, San Diego, CA (US); Wun-Ling Chang, San Diego, CA (US)

(73) Assignee: Wonder Spray, LLC, San Diego, CA (US)

(21) Appl. No.: 17/518,452

(22) Filed: Nov. 3, 2021

Related U.S. Application Data
(63) Continuation-in-part of application No. 17/179,251, filed on Feb. 18, 2021.
Application No. 17/179,251 is a continuation-in-part of application No. 16/833,209, filed on Mar. 27, 2020.
Application No. 16/833,209 is a continuation of application No. 16/293,551, filed on Mar. 5, 2019.

(51) Int. Cl. A61K 33/20; A61K 9/08; A61K 9/00; A61P 33/02; A61P 31/10; A61P 31/12; A61P 31/04

(52) U.S. Cl. CPC A61K 33/20; A61K 9/08; A61K 9/0078; A61K 9/006; A61P 31/10; A61P 31/12; A61P 31/04; A61P 33/02

(57) ABSTRACT

Systems and methods for disinfecting and deodorizing using low concentration hypochlorous acid solutions are disclosed. In one embodiment, a method may include delivering an disinfectant solution to PAP equipment, such as CPAP and BiPAP equipment, to disinfect the PAP equipment used by the patient, thereby preventing infection by the infectious diseases in the patient. The disinfectant solution may include an aqueous solution of hypochlorous acid. The hypochlorous acid may be between 30 parts per million (ppm) to 500 ppm of the aqueous solution.

### CROSS-REFERENCE TO RELATED APPLICATIONS

The present application is a continuation-in-part of U.S. patent application Ser. No. 17/179,251, filed Feb. 18, 2021, which is a continuation-in-part of U.S. patent application Ser. No. 16/833,209, filed on Mar. 27, 2020, which is a continuation of U.S. patent application Ser. No. 16/293,551, filed on Mar. 5, 2019, the contents of both which are incorporated herein by reference in their entirety.

### TECHNICAL FIELD

The present disclosure is generally related to disinfecting and deodorizing PAP equipment using hypochlorous acid (HOCI). Embodiments of the present disclosure relate to inhibiting growth of pathogens, including bacteria, viruses, yeast, mold, fungi, spores, protozoa, and/or prion in PAP equipment. Various embodiments of the present disclosure can be used for disinfection and deodorization of various medical equipment, including CPAP devices, BiPAP devices, asthma nebulizers, oxygen concentrators, medical tubing, medical hoses, and masks.

### BACKGROUND

Positive airway pressure (“PAP”) equipment is commonly used to assist with breathing while sleeping, for example, as a remedy for sleep apnea. PAP equipment is made up of various components and accessories that work together to assist with breathing while asleep. Commonly, PAP equipment includes a mask connected to a hose or tube, which is further connected to the base of a PAP device. The PAP device typically contains a motor that pressurizes room temperature air and delivers it through a hose or tube connected to a mask worn by the patient. PAP devices further contain a reservoir that is often filled with distilled water to humidify the pressurized air. PAP devices assist with breathing by keeping a constant level of pressure greater than atmospheric pressure applied to the upper respiratory tract of a person. Due to the daily use of PAP equipment, PAP equipment is known to harbor pathogens, including bacteria, viruses, yeast, mold, fungi, spores, protozoa, and/or prions. Traditionally, PAP equipment is cleaned by disassembling the PAP device, hose or tube, and mask, and then rinsing the components with a mild soap and water. The traditional cleaning method of PAP equipment can be problematic as many individuals lack the time, discipline, or technique to properly and regularly clean their PAP equipment sufficiently to prevent the growth of pathogens, including bacteria, viruses, yeast, mold, fungi, spores, protozoa, and/or prion.

Antimicrobial medications are commonly used to treat infectious disease. Antimicrobial resistance is a key issue that needs to be taken into account when selecting a therapeutic agent for the treatment of infectious diseases. For example, hospital infections due to multi-resistant bacteria, such as MRSA or Gram-negative multi-resistant bacteria, are serious threats. Although bacterial resistance is a natural phenomenon, the misuse of antimicrobial drugs has accelerated the development of resistance. Accordingly, a treatment that an infectious agent cannot develop a resistance to would be advantageous.

Antiseptic and disinfectant agents are known to destroy or inhibit the growth and development of microorganisms. Unlike antibiotics or antimicrobials that act selectively on a specific target, antiseptics and disinfectants have multiple targets and a broader spectrum of activity, which include bacteria, fungi, viruses, yeast, mold, protozoa, spores, archaea, algae, and even prions. Several antiseptic and disinfectant categories exist, including alcohols (ethanol), anilides (triclocarban), biguanides (chlorhexidine), bisphenols (triclosan), chlorine compounds, iodine compounds, silver compounds, peroxygens, and quaternary ammonium compounds. In particular, chlorine-based compounds, like bleach, have been traditionally used for antiseptic, disinfectant, and deodorization purposes, and have shown to effective in disinfecting and deodorizing PAP equipment.

### SUMMARY

Embodiments of the present disclosure are directed to systems and methods for disinfecting and deodorizing PAP equipment using low concentration hypochlorous acid solutions. One embodiment of the present disclosure relates to a method of destroying infectious agents and organisms on PAP equipment, such as continuous positive airway pressure (“CPAP”) devices, bi-level positive airway pressure (“BiPAP”) devices, asthma nebulizers, and oxygen concentrators, used by a patient. This method is used to disinfect the PAP equipment used by the patient, thereby preventing initial and continued exposure to harmful microbial from the PAP equipment. The antiseptic or disinfectant solution may include an aqueous solution of hypochlorous acid. The hypochlorous acid may be between 30 parts per million (ppm) to 500 ppm of the aqueous solution. In embodiments, the hypochlorous acid may have a pH range of approximately 4 to 7.

In some embodiments, the delivery of antiseptic or disinfectant solution may include placing the aqueous solution into a reservoir of the PAP equipment. The delivery of antiseptic or disinfectant solution may include aerosolizing the aqueous solution into particles. The delivery of antiseptic or disinfectant solution may include distributing the aerosolized particles of the aqueous solution onto the PAP equipment. In embodiments, the particles may be between 0.1 μm and 99 μm in size.

In other embodiments, the delivery of antiseptic or disinfectant solution may include placing the aqueous solution of the hypochlorous acid within a reservoir of a spray bottle. The delivery of antiseptic or disinfectant solution may include transforming the aqueous solution of the hypochlorous acid into a mist. The delivery of antiseptic or disinfectant solution may include spraying the mist of the aqueous solution of hypochlorous acid onto the PAP equipment.

In other embodiments, the delivery of antiseptic or disinfectant solution may include placing the aqueous solution of the hypochlorous acid within a reservoir of an atomizer. The delivery of antiseptic or disinfectant solution may include transforming the aqueous solution of the hypochlorous acid into a fine spray. The delivery of antiseptic or disinfectant solution may include distributing the fine spray of the aqueous solution of hypochlorous acid onto the PAP equipment.

In other embodiments, the PAP equipment may include one of CPAP devices, BiPAP devices, asthma nebulizers, oxygen concentrators, medical tubing, medical hose, and/or masks.

In further embodiments, the hypochlorous acid can be applied to medical equipment. The medical equipment may include one of medical tubing, tracheostomy tube, respiratory equipment, masks, nasal pieces, laryngoscopes, endoscopes, portable x-ray machine, ultrasound machine, echocardiogram machine, fluoroscopic equipment, blood pressure cuff, pulse oximeter, stethoscope, tracheostomies, respiratory devices, ventilators, noninvasive positive pressure ventilation devices, bronchoscopes, urinary catheter, surgical equipment, CPAP equipment, BiPAP equipment, asthma nebulizer, and oxygen concentrators.

In other embodiments, the application of antiseptic or disinfectant solution may include placing the aqueous solution of the hypochlorous acid within the reservoir of a CPAP or BiPAP device. The application of antiseptic solution may include activating the CPAP or BiPAP device and allowing the hypochlorous acid to drain through the CPAP and BiPAP equipment.

In embodiments, the application of antiseptic or disinfectant solution may include placing the aqueous solution of the hypochlorous acid within a reservoir of an atomizer. The application of antiseptic solution may include transforming the aqueous solution of the hypochlorous acid into a fine spray. The application of antiseptic or disinfectant solution may include distributing the fine spray of the aqueous solution of hypochlorous acid to the PAP equipment.

In embodiments, the particles may be approximately from 0.1 μm to 99 μm in size.

### BRIEF DESCRIPTION OF THE DRAWINGS

FIG. 1 illustrates a perspective view of an antiseptic agent delivery system for administering HOCI comprising a nebulizer, according to an implementation of the disclosure.

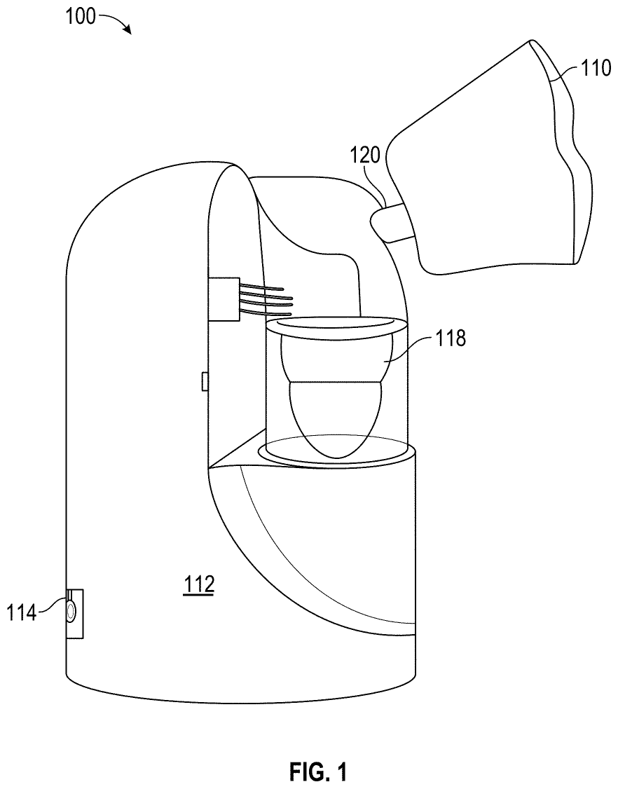

FIG. 2 illustrates a process of administering HOCI using a nebulizer, according to an implementation of the disclosure.

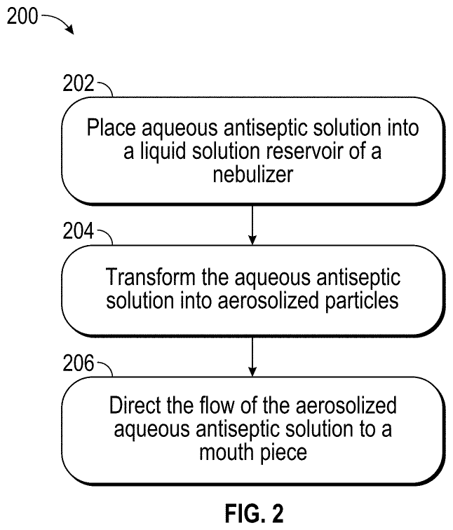

FIG. 3 illustrates tables showing the effect of a solution of HOCI on Streptococcus Pneumoniae, according to an implementation of the disclosure.

FIG. 4 illustrates tables showing the effect of a solution of HOCI on Streptococcus Pyogenes, according to an implementation of the disclosure.

FIG. 5 illustrates a perspective view of CPAP equipment with HOCI, according to an implementation of the disclosure.

### DETAILED DESCRIPTION

Hypochlorous acid (HOCI) is a weak acid that forms when sodium chloride dissolves in water, and itself partially dissociates, forming HOCL and hypochlorite, OCI-, depending on the solution pH. Similar to other chlorine-releasing agents (e.g., sodium hypochlorite, chlorine dioxide, and the N-chloro compounds such as sodium dichloroisocyanurate), aqueous HOCL is well known for its antimicrobial, anti-inflammatory, and immunomodulatory properties.

Applications of aqueous solutions containing approximately 30-2500 ppm (0.003% to 0.25%) HOCI are used in a variety of areas including (but not limited to) wound care, as antimicrobial agents, as anti-allergen agents, dental care and there are also significant applications in water treatments, food sanitization, and hard surface disinfection, and cosmetics. HOCI can also be used as a disinfectant in medical equipment, including CPAP equipment, BiPAP equipment, asthma nebulizers, oxygen concentrators, medical tubing, tracheostomy tube, respiratory equipment, masks, nasal pieces, laryngoscopes, endoscopes, portable x-ray machine, ultrasound machine, echocardiogram machine, fluoroscopic equipment, blood pressure cuff, pulse oximeter, stethoscope, tracheostomies, respiratory devices, ventilators, noninvasive positive pressure ventilation devices, bronchoscopes, urinary catheter, surgical equipment, and/or other medical equipment. HOCI can also be used therapeutically and/or prophylactically to prevent or limit the spread of infectious organisms, clear the natural flora of a known contaminating pathogen, and/or to sterilize spaces contaminated with hard to eradicate pathogens.

HOCI is a potent antimicrobial capable of eradicating bacteria including antibiotic-resistant strains, viruses, fungi, and spores. In particular, HOCI is the active component responsible for pathogen disruption and inactivation by chlorine-releasing agents (CRAB). It is understood that the OCI-ion has little effect compared to undissolved HOCI, as the hypochlorite (OCI-), has only a minute effect compared to undissolved HOCI. Accordingly, the microbicidal effect of HOCI is the greatest when the percentage of undissolved HOCI is highest. In an aqueous solution of HOCI, ranging from approximately pH 4 to pH 7, chlorine exists predominantly as HOCI, whereas above pH 9, CIO-predominates.

Because HOCI is a highly active oxidizing agent, its mode of operation comprises destroying and/or deactivating cellular activity of proteins. For example, HOCI targets bacteria by chemically linking chlorine atoms to nucleotide bases that disrupt the function of bacterial DNA, impede metabolic pathways in which cells use enzymes to oxidize nutrients, and release energy, and other membrane-associated activities. Additionally, HOCI has also been found to disrupt oxidative phosphorylation and other membrane-associated activity. Similarly, HOCI has been found to inhibit bacterial growth. For example, at 50 mM (2.6 ppm), HOCI completely inhibited the growth of E. coli within 5 minutes, including inhibiting the DNA synthesis by ninety-six percent. Unlike conventional antibiotics, the antimicrobial activity of HOCI is directly toxic to microbial cells, including many Gram-positive and Gram-negative bacteria and their biofilms. HOCI has demonstrated disinfection efficacy against eradication of bacteria, including Acinetobacter baumannii, Bacillus subtilis, Enterobacter cloacae, Enterococcus faecalis, Escherichia coli, Enterobacter, Klebsiella pneumoniae, Listeria monocytogenes, MRSA (Staph. aureus), Polymicrobial biofilm, Proteus vulgaris, Pseudomonas aeruginosa, Salmonella choleraesuis, Shigella flexneri, Staph epidermidis, and Yersinia enterocolitica. HOCI can also be used to treat, kill, disinfect, minimize levels of, and/or otherwise affect infectious disease agents, infectious diseases, and/or complications after infections, including mast cell degranulation, acne, pneumonia, streptococcus pyogenes, biofilms, bronchiectasis, asthma, acute respiratory distress syndrome (ARDS), bronchitis, sleep apnea, chronic obstructive pulmonary disease (COPD), chest infections, cystic fibrosis, tuberculosis, liver cirrhosis, staphylococcus aureus, haemophilius influenzae, klebsiella pnuemoniae, pseudomona aeruginosa, bordetella pertussis, moraxella catarrhalis, coxiella burnetiid, chlamdyophilia pneumoniae, mycoplasma pneumoniae, legionella pneumophilia, yesinia pestis, influenza viruses, rhinoviruses, respiratory syncytial virus, adenovirus, enterovirus, parainfluenza, Epstein-Barr virus, cytomegalovirus, hantavirus, Herpes simplex, histoplasma capsulatum, blastomyces, pneumocystis, coccidiodes, thrush, herpes simplex ulcers, other infections of the mouth, otitis media, cavity-causing bacteria, gingivitis, Helicobacter pylori, Giardia, tapeworms, Entamoeba, other GI-infecting organisms, Clostridium difficile, colitis, diarrhea, Candida, vaginitis, drug-resistant bacteria, pruritic, SARS-CoV-2, and the like. HOCI can also be used as a prophylaxis for splenectomized patients and others at risk for pneumonia, which is a commonly found risk for patients who regularly use PAP equipment.

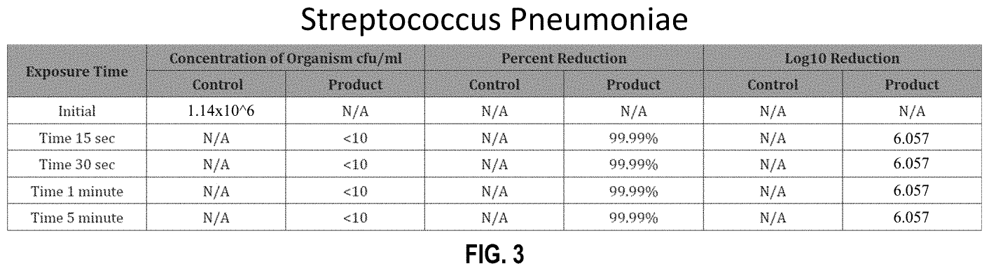

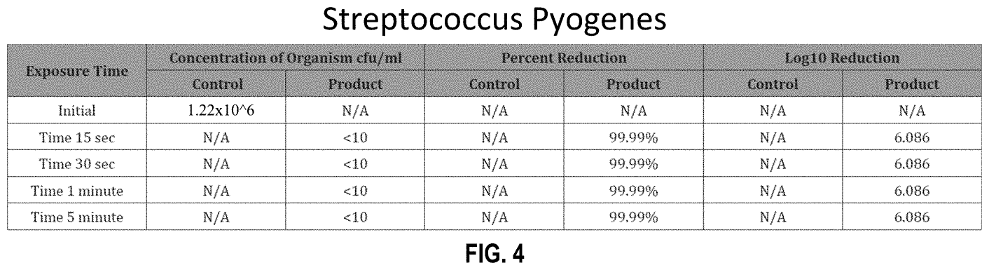

For example, FIGS. 3 and 4 illustrate tables of the effect of a solution of HOCI on Streptococcus pneumoniae and Streptococcus pyogenes, respectively. The organisms in this test were prepared by inoculating the surface of about 5% Sheep blood agar plates, incubated at about 30 to 35° C. for 18 to 24 hours. Following the incubation period, the plates were washed with sterile Serological Saline Solution to harvest the microorganisms used and dilutions with Saline were made, plated on blood agar and incubated at 30 to 35° C. for 24-48 hours to determine the concentration. The inoculum level was then adjusted to 108 cfu/ml for use as a stock suspension. Stock suspensions were well mixed and homogenized at each inoculation interval. The following microorganisms were used in this Kill Time Study to demonstrate the antimicrobial properties of the HOCI solution against common pathogenic organisms: Microbiologies Kwik-Stik Streptococcus pyogenes ATCC 49399, Streptococcus pneumoniae ATCC 49619.

Positive controls were performed at initiation and completion by spread plating to enumerate inoculum levels and verify culture purity during testing and Negative controls were performed to establish sterility of media, reagents, and materials used at initiation. Neutralizer Suitability using Modified Letheen Broth (MLB) was performed concurrently with Kill Time testing to confirm the recovery of <100 CFU of the test organism in the subculture media in the presence of product. Duplicate 10 ml containers for each treated specimen or material concentration was prepared, equilibrated to 25±2° C., and 0.1 ml of inoculum is added to each container to achieve a final concentration of 106 cfu/ml.

Serial dilutions from each replicate were made at intervals of 15 second, 30 second, 1 minute and 5 minutes using 1 ml of the inoculated test product into 9 ml MLB from 1:10 to 1:1000000. Subsequently, 1 ml from each dilution was spread plated on 5% Sheep Blood agar plate in duplicate, incubated at 30 to 35° C. for 48 hours. After the incubation period, all plates were counted to determine the number of microorganisms, results are averaged and reported as log 10 reductions.

Referring back to FIG. 3, after fifteen seconds, there were less than 10 cfu/ml of the Streptococcus Pneumoniae after the HOCI solution was applied, the percent reduction for Streptococcus Pneumoniae was 99.99% after the HOCI solution was applied, and the log 10 reduction for Streptococcus Pneumoniae was 6.057 after the HOCI solution was applied.

In FIG. 4, after fifteen seconds, there were less than 10 cfu/ml of the Streptococcus Pyogenes after the HOCI solution was applied, the percent reduction for Streptococcus Pyogenes was 99.99% after the HOCI solution was applied, and the log 10 reduction for Streptococcus Pyogenes was 6.086 after the HOCI solution was applied.

FIGS. 3 and 4 illustrate that the bacteria that cause strep throat and pneumonia are effectively killed by HOCL in less than 15 seconds. The currently disclosed solution of HOCI overcomes the issues with other medicine commonly prescribed for these diseases, and is an effective prophylaxis for those using PAP equipment against these diseases. For example, healthcare professionals may prescribe penicillin or amoxicillin to treat these bacterial infections. Side effects of penicillin antibiotics include diarrhea, dizziness, heartburn, insomnia, nausea, itching, vomiting, confusion, abdominal pain, easy bruising, bleeding, rash, and allergic reactions. The most common side effects of amoxicillin are nausea, vomiting, stomach pain and diarrhea. Moreover, amoxicillin may also destroy the good bacteria that naturally resides in the body. This can lead to an overgrowth of yeast, which may not only produce diarrhea but also cause yeast infections, especially in the mouth and vagina. In addition, oral amoxicillin exposure may cause shifts in microbiome composition that can last approximately 30 days on average and more than 2 months in some individuals. This shift in microbiome composition can also predispose patients to Clostridium difficile colitis. The additional concern about resistance to antibiotics is well known. Bacteria have not been known to develop a resistance to the HOCL nor is it known to cause any of the side effects listed above.

Additionally, HOCI possesses viricidal activity properties. For example, it has been demonstrated that HOCI inactivated naked f2 RNA at the same rate as RNA in intact phage, whereas f2 capsid proteins could still adsorb to the host. HOCI has demonstrated disinfection efficacy against eradication of viruses including norovirus, filoviruses such as Ebola, and human coronaviruses like MERS-CoV, SARS, and SARS-CoV-2, as well as fungi such as Candida and Aspergillus. Further, as a sporicide, HOCI causes the spore coat to detach from the cortex, where further degradation occurs.

Both topical and internal applications of HOCI are safe because it is the exact same substance white blood cells in the human body produce to fight infection. Indeed, extensive studies have demonstrated exceptional safety of HOCI. The Food and Drug Administration (FDA) has cleared preparations of HOCI to be used, e.g., topically for wound cleansing, eye infections, tooth infections, nasal decontamination, and the care of surgical incisions. In particular, inhaling the aerosolized form of HOCI has also been shown to causes no adverse effects.

The advent of antibiotics and other area disinfectants led to a reduction in environmental use of HOCI. However, widespread use of antibiotic agents led to antimicrobial resistance. Accordingly, an urgent need to optimize currently available anti-infectious therapies to overcome drug resistance exists. Antimicrobial resistance has not been observed for HOCI.

Positive airway pressure (“PAP”) devices keep patients airways open by providing a continuous stream of air through a tube or hose connected to a mask. There are several types of PAP devices, but the two primary forms are continuous positive airway pressure (“CPAP”) devices and bi-level positive airway pressure (“BiPAP”) devices. A CPAP device can be set to a constant pressure that will be maintained throughout the operation of the device, typically be a person sleeping with sleep apnea. A BiPAP device is similar to a CPAP device except that the pressure can be set to two different pressures that will change. For example, on a BiPAP device a patient will set a high-ipap pressure for inhalation and a low-epap pressure for exhalation.

Both CPAP and BiPAP devices are constructed with having a motorized component that pressurizes air within the device, a reservoir that humidifies the pressurized air, and a regulator component that can set the pressure at which the device is operated. A BiPAP device often has two regulators that allow for different pressures on inhalation and exhalation, as explained above. Typically, patients using a CPAP or BiPAP device will connect the device to a tube or hose made from medical rubber or medical plastic, and then subsequently fasten the hose or tube to a rubber or plastic mask. The mask is then be secured to patient's face and head, where the device will apply constant, pressurized airflow to the patient during the time of wearing the mask while the CPAP or BiPAP device is operational.

A primary concern on behalf of patients who use a CPAP or BiPAP device is the risk of buildup of microorganisms and/or other pathogens, including bacteria, viruses, yeast, mold, fungi, spores, protozoa, and/or prions. The buildup of unwanted microorganisms can be the result of patients breathing into the device, the device being exposed to microorganisms when not in use, and due to excessive moisture accumulation. It is a common concern for CPAP and BiPAP users to be able to properly clean their equipment, and traditional methods such as disassembling the equipment and submerging the equipment in a mild soap with water, requires constant attention and is known to be ineffective at fully cleaning the equipment.

Various embodiments of the method disclosed herein are directed to antiseptic and disinfectant agent delivery systems in which the disinfectant is distributed via aerosolization or atomization of hypochlorous acid within CPAP and BiPAP devices. For example, an aqueous solution containing hypochlorous acid can be placed in the reservoir of a CPAP or BiPAP device where the patient would usually place distilled water. The CPAP or BiPAP device may be run either with the patient using the CPAP or BiPAP machine for one to ten minutes, which provides a dual function of both cleaning the CPAP or BiPAP device and acting as a nebulizer for the patient, or the CPAP or BiPAP machine can be run for one to thirty minutes without the patient using the mask to disinfect and deodorize the device.

When a CPAP or BiPAP machine is run with hypochlorous acid and the patient is wearing the mask connected to the device, the hypochlorous acid functions as both a disinfectant for the device and an antiseptic for the patient. When the CPAP or BiPAP machine is run with hypochlorous acid and the patient is disconnected from the device, the hypochlorous acid functions solely as a disinfectant and deodorizer for the device.

Various embodiments of the method disclosed herein are directed to antiseptic and disinfectant agent delivery systems in which the antiseptic or disinfectant agent is administered via a pulmonary route as a treatment of infectious diseases caused by microbes (e.g., bacteria, spores), viral, fungal, allergy-causing agents. Because the inhalation process gives a more direct access to the target organ/cavity than more traditional routes (e.g., topical, oral, intravenous, etc.), the pulmonary administration of HOCI used to inhibit viral and bacterial growth provides a therapeutic approach that may help avoid reduce antimicrobial resistance while alleviating the disease symptoms. For example, upper respiratory tract infections caused by one or more bacterial or viral pathogens such as bronchitis, epiglottitis, laryngitis, sinusitis, rhinosinusitis, chronic rhinosinusitis and so on, lung infections, such as pneumonia, may be treated by a pulmonary administration of an antiseptic agent, such as HOCI.

In some embodiments, a solution of HOCI may be delivered via the pulmonary route utilizing a number of pulmonary delivery devices. For example, the HOCI may be delivered via a CPAP device, BiPAP device, asthma nebulizer, oxygen concentrator, aerosolizer, atomizer, and/or any other such pulmonary delivery device. For example, a solution of HOCI of low concentration levels and relatively low acidotic pH may be used as a nebulized topical laryngeal, tracheal, and alveolar disinfectant. In some embodiments, the aqueous solution of HOCI may include a concentration of approximately 0.02 percent of HOCI dissolved in water.

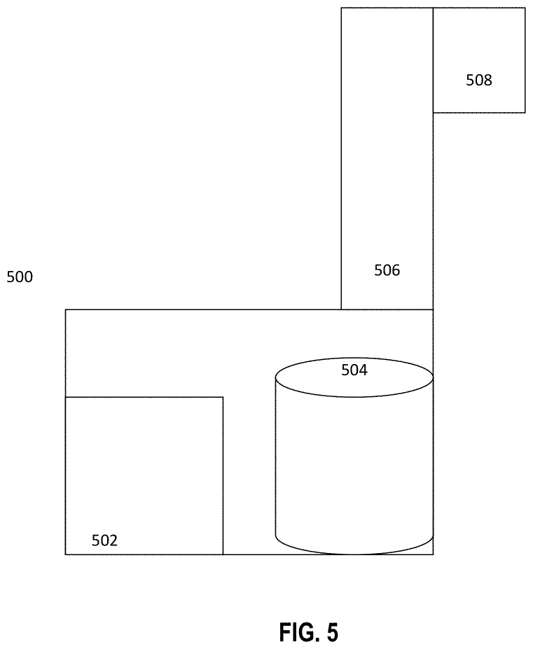

FIG. 5 depicts HOCI within CPAP equipment for disinfecting and deodorizing the CPAP equipment. The CPAP machine 500 includes a liquid reservoir 504 and a motorized component 502 that atomizes that liquid in the reservoir 504 . In some embodiments, the reservoir 504 can be filled with HOCI to be atomized and disinfect and deodorize the CPAP machine 500 as described herein. In other embodiments, the HOCI can be atomized to disinfect and deodorize the tubing/hose 506 or the mask 508 worn by the patient, as described herein.

As shown in FIG. 5, the CPAP equipment comprises a motorized component 502 which atomizes or humidifies the liquid, for example distilled water, for another example an aqueous solution containing HOCI. For example, the aqueous disinfectant solution may include liquid HOCI liquid solution ranging from 0.5 ml to 20 ml placed in the reservoir 104. Further, the CPAP equipment comprises a hose/tube 506 that carries humidified or atomized air to the mask 108 from the CPAP machine 500. The mask 508 is commonly worn by patients to inhale/exhale the humidified or atomized air.

In some embodiments, the antiseptic solution may be diffused. The diffusion may be the result of nebulization, ultrasonication, and/or other mechanisms accomplished through PAP equipment. A nebulizing PAP effect may send air through a PAP device, followed by a tube or hose and mask.

In some embodiments, illness suspected to be caused by adverse reactions to one or more medications and/or lifestyle choices may be treated by administering HOCI through the above described pulmonary delivery methods. For example, oral inflammation and/or ulceration (e.g., mucositis) which may arise as an adverse effect to a particular medication (e.g., chemotherapy and radiotherapy treatment for cancer) or due to dehydration, poor mouth care, oxygen therapy, excessive use of alcohol and/or tobacco, and lack of protein in the diet may be treated by placing a particular amount of aqueous solution of HOCI (e.g., approximately 5 mL) may administered by the above described pulmonary delivery methods. In some embodiments, the aqueous solution of HOCI may be diluted with one or more diluents. For example, approximately 0.5 ml to 20 ml of saline may be added to the aqueous solution of HOCI.

In some embodiments, illness suspected to be caused by microbes, bacteria, spores, viral, fungal, and/or allergy-causing agents may be treated by administering HOCI through administering the above described pulmonary delivery method.

In some embodiments, illness suspected to be caused microbes, bacteria, spores, viral, fungal, and/or allergy-causing agents may be treated by administering HOCI through the above described pulmonary delivery method.

In some embodiments, one or more effects of relaxing one or more respiratory structures (e.g., uvula, soft palate, etc.) resulting in a sound (e.g., snoring) due to their vibrations during sleep may be treated by administering HOCI. In some embodiments, the HOCI solution may be administered by a pulmonary delivery method, as described above.

In some embodiments, an irritation, inflammation, and/or obstruction of the breathing passages resulting in a cough reflex and often associated with acute and/or chronic respiratory tract infection may be treated by administering HOCI. In some embodiments, the HOCI solution may be administered by a pulmonary delivery method, as described above.

In some embodiments, an irritation and/or an inflammation of the voice box resulting in loss of voice and/or diminished capacity to produce sound (e.g., laryngitis) may be treated by administering HOCI. In some embodiments, the HOCI solution may be administered by a pulmonary delivery method, as described above.

In some embodiments, an irritation and/or an inflammation of one or more structures within the nasal cavity and/or throat due to an allergic reaction to one or more allergens, such as pet dander, dust, mites, pollen and mold, may be treated by administering HOCI. In some embodiments, the HOCI solution may be administered by a pulmonary delivery method, as described above. Alternatively, the HOCI solution may be used to decrease the histamine response which may be elevated during an allergic response to one or more allergens, as previously alluded. For example, the HOCI may be administered through the use of a HOCI containing oral rinse solution, a gargling solution, as described above, or a pulmonary delivery method.

In some embodiments, infectious disease agents, infectious diseases, and/or complications after infections (e.g., diseases caused by microbes (including spores), antimicrobes, pollutants, microorganisms, biofilms, viruses, bacteria, fungi, protists, parasites, allergy-causing agents, and/or other organisms, including mast cell degranulation, acne, pneumonia, biofilms, bronchiectasis, asthma, acute respiratory distress syndrome (ARDS), bronchitis, sleep apnea, chronic obstructive pulmonary disease (COPD), chest infections, cystic fibrosis, tuberculosis, liver cirrhosis, staphlococcus aureus, haemophilius influenzae, klebsiella pnuemoniae, pseudomona aeruginosa, bordetella pertussis, moraxella catarrhalis, coxiella burnetiid, chlamdyophilia pneumoniae, mycoplasma pneumoniae, legionella pneumophilia, yesinia pestis, influenza viruses, rhinoviruses, respiratory syncytial virus, adenovirus, enterovirus, parainfluenza, Epstein-Barr virus, cytomegalovirus, hantavirus, Herpes simplex, histoplasma capsulatum, blastomyces, pneumocystis, coccidiodes, thrush, herpes simplex ulcers, other infections of the mouth, otitis media, cavity-causing bacteria, gingivitis, Helicobacter pylori, Giardia, tapeworms, Entamoeba, other GI-infecting organisms, Clostridium difficile, colitis, diarrhea, Candida, vaginitis, drug-resistant bacteria, pruritic, and the like) present, or capable of spreading, on medical equipment (e.g., medical tubing, tracheostomy tube, respiratory equipment, masks, nasal pieces, laryngoscopes, endoscopes, portable x-ray machine, ultrasound machine, echocardiogram machine, fluoroscopic equipment, blood pressure cuff, pulse oximeter, stethoscope, tracheostomies, respiratory devices, ventilators, noninvasive positive pressure ventilation devices, bronchoscopes, urinary catheter, surgical equipment, CPAP equipment, BiPAP equipment, asthma nebulizer, oxygen concentrators, and/or other medical equipment) used by patients may be disinfected and/or cleaned by administering HOCI to the medical equipment through the use of various delivery mechanisms, such as placing the HOCL in the reservoir of a CPAP machine and then running the machine, thereby prophylactically treating patients from the infectious diseases. The treatment may be administered, or delivered, for 5 seconds, 15 seconds, 30 seconds, 1 minute, 1 hour, 6 hours, 12 hours, etc. or any time period in between. It should be appreciated that in some embodiments the amount of solution administered, or delivered, is limited by the delivery mechanism (e.g., a spray into a nasal cavity is limited by the spray bottle and related components).

The delivery mechanisms may include bathing the PAP equipment in an aqueous solution with HOCI, rinsing the PAP equipment in an aqueous solution with HOCI, spraying the PAP equipment in an aqueous solution with HOCI, misting the PAP equipment with an aqueous solution with HOCI, fogging the PAP equipment in an aqueous solution with HOCI, aerosolizing the PAP equipment with an aqueous solution with HOCI, electrostatically spraying the PAP equipment with an aqueous solution with HOCI, placing HOCL within the PAP equipment and operating the equipment to disseminate the aqueous solution with HOCL, and/or other delivery mechanisms.

For example, spraying the PAP equipment with an aqueous solution with HOCI may include pumping a trigger on a container that lowers the air pressure within the tube that is in the bottle. The pumping action forces the solution up the tube into a smaller nozzle that is converted into a spray or a mist.

Similarly, misting the PAP equipment with an aqueous solution with HOCI may include using a pressure pump to send a solution through a nozzle to turn the solution in a mist. Further, fogging the PAP equipment with an aqueous solution with HOCI may include thermal foggers, ULV, or cold, foggers, and/or other foggers. Thermal foggers may use heat to vaporize a solution to be sprayed as a fog. The resulting particle size may be between about 0.5 to 10 microns. ULV foggers may use motors that produce a high power, low pressure air stream. The solution may be sent through the air stream through a nozzle that gives the resulting air flow a swirling motion separating it into tiny particles. The resulting particle size may be between about 5 to 30 microns.

Aerosolizing the PAP equipment in an aqueous solution with HOCI may include pressurizing the solution within a container and using a propellant to push the solution into the air in aerosolized form and then what, are you spraying the pressurized solution onto the equipment, if so, then state that. For example, aerosolizing PAP equipment (e.g., CPAP device, tubes, and mask) with an aqueous solution with HOCL and then what, finish this thought.

Additionally, operating PAP equipment with an aqueous solution with HOCL such that the HOCL is distributed throughout the machine to disinfect and deodorize the PAP equipment.

In some embodiments, infectious disease agents, infectious diseases, and/or complications after infections (e.g., diseases caused by microbes (including spores), antimicrobes, pollutants, microorganisms, biofilms, viruses, bacteria, fungi, protists, parasites, allergy-causing agents, and/or other organisms, including mast cell degranulation, acne, pneumonia, biofilms, bronchiectasis, asthma, acute respiratory distress syndrome (ARDS), bronchitis, sleep apnea, chronic obstructive pulmonary disease (COPD), chest infections, cystic fibrosis, tuberculosis, liver cirrhosis, staphlococcus aureus, haemophilius influenzae, klebsiella pnuemoniae, pseudomona aeruginosa, bordetella pertussis, moraxella catarrhalis, coxiella burnetiid, chlamdyophilia pneumoniae, mycoplasma pneumoniae, legionella pneumophilia, yesinia pestis, influenza viruses, rhinoviruses, respiratory syncytial virus, adenovirus, enterovirus, parainfluenza, Epstein-Barr virus, cytomegalovirus, hantavirus, Herpes simplex, histoplasma capsulatum, blastomyces, pneumocystis, coccidiodes, thrush, herpes simplex ulcers, other infections of the mouth, otitis media, cavity-causing bacteria, gingivitis, Helicobacter pylori, Giardia, tapeworms, Entamoeba, other GI-infecting organisms, Clostridium difficile, colitis, diarrhea, Candida, vaginitis, drug-resistant bacteria, pruritic, and the like) may prevent infection, therapeutically treat the infectious diseases, kill the pathogen causing the infectious disease, and/or limit progression of the infectious diseases by administering HOCI through use of various delivery mechanisms.

The delivery mechanisms may include ingestion of an aqueous solution with HOCI to a patient via medical equipment, rinsing the affected area on and/or in the patient with the aqueous solution of HOCI (e.g., neti pot, liquid rinse, etc.), spraying the aqueous solution with HOCI onto or into the patient (e.g., nasal spray, throat spray, ear spray, etc.), misting the aqueous solution with HOCI onto or into the patient (e.g., nasal mist, throat mist, ear mist, etc.), fogging the aqueous solution with HOCI onto or into the patient, aerosolizing the aqueous solution with HOCI onto or into the patient, electrostatic, operating CPAP equipment with the aqueous solution with HOCL onto or into the patient, and/or other delivery mechanisms. It should be appreciated that the location where the HOCI solution is delivered may be different depending on the particular infectious disease. For example, treating an allergy-causing agent may be sprayed into a nasal cavity, while treating asthma may be sprayed into the mouth and/or throat. In another example, treating acne may be a liquid application to the affected area. As one example, infections of the upper sinuses may be prevented or prophylactically treated by delivering a HOCI solution via a nasal spray or rinse at about 100 ppm. Patients with recurrent sinusitis may use HOCI to prevent or prophylactically treat the sinusitis. HOCI may be used to prevent or prophylactically treat patients with severe allergies that often progress to sinusitis. HOCI via a nasal spray or rinse at least once per day at about 100 pm treatment may be used to (i) prevent infection, (ii) kill the pathogen in the early stages shortly after exposure, and/or (iii) limit progression from the sinuses to the lung. HOCI may be used to prophylactically treat an exposed or at-risk individual, thereby preventing infection, killing the pathogen early, and/or preventing migration to the lung.

In some embodiments, infections of the lungs and/or bronchitis may be prevented or prophylactically treated by using a PAP device containing an aqueous solution of HOCI at least once per day at 100 pm of HOCI. Prophylaxis using HOCI may benefit following groups at risk for recurrent lung infections:

Immunosuppressed (e.g., due to hereditary immunodeficiency, HIV, chemotherapy, biologic therapy (e.g. anti-TNF), transplant, etc.)—These patients may be at a higher risk for pneumonia due to organisms that do not cause pneumonia in immunologically intact patients, as well as routine respiratory pathogens.

Asplenectics—Patients who have had their spleens removed may be susceptible to lung infections by pathogens, such as Streptococcus pneumoniae.

Cirrhosis—Patients with cirrhosis may be susceptible to lung infections by pathogens, such as S. pneumoniae.

Chronic lung diseases (e.g., cystic fibrosis, asthma, lung cancer, chronic obstructive pulmonary disease [COPD], chronic interstitial lung diseases, chronic pulmonary fibrosis, etc.)—Patients with chronic lung diseases may be at risk for pneumonia.

Autoimmune diseases—Autoimmune diseases (e.g., granulomatosis with granulomatous polyangiitis (GPA, formerly Wegner's Granulomatosis)) may affect the lungs and render it susceptible to infection. In these situations, the HOCI solution may be used to provide anti-inflammatory effects.

Contamination of respiratory assist devices—Tracheostomies, noninvasive positive pressure ventilation devices, ventilators, CPAP equipment, BiPAP equipment, asthma nebulizer, oxygen concentrators, etc. may become colonized with organisms (e.g., some that form biofilms within the tubing), and these organisms may infect the lungs and lead to pneumonia. As one example of minimizing the likelihood of organism colonization of the medical equipment, nebulized HOCI (e.g., about 100 ppm) may be applied directly into a respiratory assistance devices, thereby preventing the development of nosocomial pneumonia in patients using the respiratory assistance devices. As another example of minimizing the likelihood of organism colonization of medical equipment, HOCL may be used in place of distilled water in CPAP equipment, BiPAP equipment, asthma nebulizer, and oxygen concentrators to deodorize the equipment and disinfect both the equipment and patient's respiratory system. In some embodiments, HOCI can also be used to clean the masks and/or nasal pieces of the respiratory assist devices, such as CPAP equipment, BiPAP equipment, asthma nebulizer, and oxygen concentrators, as well as devices which can be used to treat pulmonary infections.

As yet another example, infections of upper sinuses and sinusitis may be treated by using a PAP device with aqueous solution of HOCI once per day.

As another example, infections of the lungs and bronchi may be treated by PAP device containing an aqueous solution of HOCI once per day. In some embodiments, HOCI may assist antibiotic therapy in clearing or decreasing the organism load from the lung cavity.

As another example, HOCI may be used to clean PAP equipment that may be contaminated by organisms, some of which form biofilms. Bacterial biofilms are a source of infection when used for diagnostics and/or treatment. HOCI may be used to penetrate and kill bacteria within biofilms. HOCI may be in a misted or soaked medium to decontaminate the PAP equipment (e.g., CPAP devices). Misted HOCI may also be used to decontaminate reused masks.

In some embodiments, the pH level of the HOCI solution administered through the methods disclosed herein may be pH-neutral because stabilized and/or pH-neutral HOCI is superior in terms of antimicrobial activity to non-stabilized HOCI and acidified bleach, including against hypochlorite-resistant strains. In some embodiments, the acidotic pH level of the HOCI may be within the range resulting in the highest amount of undissolved HOCI. For example, the acidotic pH level may range from approximately pH 6.1 to approximately pH 6.3. In another example, the acidotic pH level may range from approximately pH 4.0 to approximately pH 7.0.

In some embodiments, HOCI solution may be placed in the reservoir of a CPAP device to disinfect and deodorize the CPAP device, tubing, and mask, collectively the CPAP equipment. For example, the HOCI solution may be placed in the reservoir of the CPAP device and the CPAP device can be run without the patient wearing the mask for any amount of time ranging from one to thirty minutes. In another example, HOCI solution may be placed in the reservoir of the CPAP device and the CPAP device can be run with the patient wearing the mask for any amount of time ranging from one to thirty minutes.

In some embodiments, a mist comprising an aqueous HOCI solution may include one or more of the following: placing an aqueous solution of HOCI ranging from 0.1 ml to 10 ml into a spray bottle adequately suited for disinfecting an deodorizing, and administering the mist comprising aqueous solution HOCI into the mask, tubing, or CPAP device of the CPAP equipment by compressing the spray bottle. For example, approximately one to ten sprays may be administered into the mask for CPAP equipment by compressing the spray bottle. In another example, approximately one to ten sprays may be administered into or upon the tubing of the CPAP equipment by compressing the spray bottle. In another example, approximately one to ten sprays may be administered into or upon the CPAP device and CPAP device reservoir by compressing the spray bottle. In some embodiments, the aqueous solution of HOCI may be diluted with one or more diluents. For example, approximately 10 ml of saline may be added to 10 mL of aqueous solution of HOCI.

In some embodiments, the aforementioned method for cleaning CPAP equipment can be used on BiPAP equipment, asthma nebulizers, and oxygen concentrators, and/or such similar pulmonary devices.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. Moreover, the use of patients, individuals, and similar terms may refer to humans and animals.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A method of disinfecting and deodorizing PAP equipment using low concentration hypochlorous acid, the method comprising:
delivering a disinfectant solution to the PAP equipment to disinfect the PAP equipment used by a patient;
wherein the disinfectant solution comprises an aqueous solution of hypochlorous acid, and wherein the hypochlorous acid is between 30 parts per million (ppm) to 500 ppm of the aqueous solution.

2. The method of claim 1, wherein the hypochlorous acid has a pH range of approximately 4 to 7.

3. The method of claim 1, wherein delivering the disinfectant solution comprises:
placing the aqueous solution into a reservoir of the PAP equipment;
aerosolizing the aqueous solution into particles; and
distributing the aerosolized particles of the aqueous solution onto and within the PAP equipment.

4. The method of claim 3, wherein the aerosolized particles of the aqueous solution are between 0.1 μm and 99 μm in size.

5. The method of claim 1, wherein delivering the disinfectant solution comprises:
placing the aqueous solution of the hypochlorous acid within a reservoir of a spray bottle;
transforming the aqueous solution of the hypochlorous acid into a mist; and
spraying the mist of the aqueous solution of hypochlorous acid onto and within the PAP equipment.

6. The method of claim 1, wherein delivering the disinfectant solution comprises:
placing the aqueous solution of the hypochlorous acid within a reservoir of an atomizer;
transforming the aqueous solution of the hypochlorous acid into a fine spray; and
distributing the fine spray of the aqueous solution of hypochlorous acid onto the PAP equipment.

7. The method of claim 1, wherein the PAP equipment is selected from the group consisting of: CPAP devices, BiPAP devices, asthma nebulizers, oxygen concentrators, medical tubing, PAP tubing, and masks.

8. A method of prophylactically treating infectious diseases, the method comprising:
applying an antiseptic solution to medical equipment used by a patient, thereby preventing infection by the infectious disease to the patient;
wherein the antiseptic solution comprises an aqueous solution of hypochlorous acid, and wherein the hypochlorous acid is between 30 ppm to 500 ppm of the aqueous solution.

9. The method of claim 8, wherein the hypochlorous acid has a pH range of approximately 4 to 7.

10. The method of claim 8, wherein applying the antiseptic solution comprises:
placing the aqueous solution of the hypochlorous acid within a reservoir of an atomizer;
transforming the aqueous solution of the hypochlorous acid into a fine spray; and
distributing the fine spray of the aqueous solution of the hypochlorous acid to the medical equipment.

11. The method of claim 8, wherein the medical equipment comprises one of CPAP equipment, BiPAP equipment, asthma nebulizers, oxygen concentrators, medical tubing, tracheostomy tube, respiratory equipment, masks, nasal pieces, laryngoscopes, endoscopes, portable x-ray machine, ultrasound machine, echocardiogram machine, fluoroscopic equipment, blood pressure cuff, pulse oximeter, stethoscope, tracheostomies, respiratory devices, ventilators, noninvasive positive pressure ventilation devices, bronchoscopes, urinary catheter, surgical equipment, asthma nebulizer, and oxygen concentrators.
